(19) United States
(12) Patent Application Publication
(10) Pub. No.: US 2002/0123469 A1
(43) Pub. Date: Sep. 5, 2002

(54) Antiproliferative colchicine compositions and uses thereof

(76) Inventor: Dennis M. Brown, Menlo Park, CA (US)

Correspondence Address: FLEHR HOHBACH TEST ALBRITTON & HERBERT LLP, Suite 3400, Four Embarcadero Center, San Francisco, CA, 94111-4187, US

(21) Appl. No.: 09/996,354

(22) Filed: Oct. 31, 2001

Related U.S. Application Data
(60) Provisional application No. 60/244,765, filed on Oct. 31, 2000.
(60) Provisional application No. 60/244,910, filed on Oct. 31, 2000.
(60) Provisional application No. 60/244,911, filed on Oct. 31, 2000.
(60) Provisional application No. 60/244,912, filed on Oct. 31, 2000.
(60) Provisional application No. 60/244,913, filed on Oct. 31, 2000.

(51) Int. Cl. A61K 31/704; A61K 31/4745; A61K 31/337

(52) U.S. Cl. 514/25; 514/283; 514/449; 514/649

(57) ABSTRACT

A method of treatment of a host with a cellular proliferative disease, including contacting the host with a colchicine family member and an antiproliferative agent, each in an amount sufficient to modulate said cellular proliferative disease, is described. In some embodiments, the colchicine family member is (S)-N-(5,6,7,9-tetrahydro-1,2,3,10-tetramethoxy-9-oxobenzo[a]heptalen-7-yl)acetamide. Antiproliferative agents of the invention include agents that interact with nucleic acids, for example, etoposide, camptothecin, and cisplatin. Antiproliferative agents of the invention also include agents that interact with tubulin targets, for example, paclitaxel and vinblastine. The invention also includes compositions containing a colchicine family member and an antiproliferative agent.

This application claims the benefit of U.S. Provisional Application Nos. 60/244,765, 60/244,910, 60/244,911, 60/244,912, and 60/244,913, each filed on Oct. 31, 2000.

### FIELD OF THE INVENTION

The technical field of the invention is the use of colchicine family members with antiproliferative agents to treat a host with a cellular proliferative disease.

### BACKGROUND OF THE INVENTION

Microtubules are involved in many important cellular functions such as cell division, cell motility, secretion, ciliary and flagellar movement, intracellular transport, and the maintenance of cell shape. Agents that interfere with mitotic spindle function likewise inhibit mitosis. Such agents are sometimes referred to as “antimitotic agents.”

Many classes of chemical compounds control microtubule assembly/disassembly by binding to tubulin. Virtually all of the observed therapeutic as well as toxic effects of the antimitotic drugs may be attributed to their actions on microtubule assembly and the subsequent microtubule-mediated processes.

Of the best characterized antimitotic agents, only paclitaxel and the vinca alkaloids such as vincristine, vinblastine and vinorelbine are currently approved as anticancer drugs. The use of agents for targeting the colchicine binding site of tubulin, in particular colchicine, remain unexploited as anticancer medicines. For example, colchicine, an antiinflammatory agent, is mainly used in the treatment of gouty arthritis.

Conventional cancer chemotherapies utilize agents from a variety of chemical classes having antiproliferative activity. There is considerable interest in modulating the efficacy of currently used antiproliferative agents to increase the rates and duration of antitumor effects in conventional antineoplastic therapies.

Topoisomerase inhibitors and cisplatin are important antiproliferative agents for cancer chemotherapy. The clinical activity of topoisomerase inhibitors and cisplatin against a number of types of cancers are demonstratable. However, improvements in tumor response rates, duration of response and ultimately patient survival are still sought. One aspect of the invention described herein is the novel use of DNA targeting agents to potentiate the antitumor effects of chemotherapeutic drugs, including cisplatin and topoisomerase I and II inhibiting agents, in particular, etoposide and camptothecins.

Additionally, taxanes and vinca alkaloids, agents which are believed to share a binding site on tubulin, demonstrate antiproliferative activity against a number of cancers. Again, however, improvements in tumor response rates, duration of response and ultimately patient survival are still sought. Thus, another aspect of the invention described herein is the novel use of colchicine, colchicine analogs, and other agents which bind to the colchicine binding site of beta-tubulin, to control tumor growth in a therapeutic treatment regimen with other tubulin targeting agents, such as the taxane, paclitaxel, and the vinca alkaloids, vinblastine and vincristine.

### SUMMARY OF THE INVENTION

Methods and compositions are provided for the treatment of a host with a cellular proliferative disease, particularly a neoplasia. In the subject methods, a pharmaceutically acceptable colchicine family member and an antiproliferative agent are administered in an amount sufficient to modulate the cellular proliferative disease.

In one aspect of the invention, the colchicine family member comprises colchicine, (S)-N-(5,6,7,9-tetrahydro-1,2,3,10-tetramethoxy-9-oxobenzo[a]heptalen-7-yl)acetamide; in another aspect, the colchicine family member comprises an analog thereof.

The antiproliferative agent of the invention may comprise an agent that interacts with nucleic acids. Alternatively, the antiproliferative agent may comprise an agent that interacts with tubulin targets.

In some aspects of the invention, the antiproliferative agent comprises taxanes, vinca alkaloids or a tubulin targeted agent. In other aspects, the antiproliferative agent comprises paclitaxel. In still other aspects, the antiproliferative agent comprises vinblastine.

Alternatively, the antiproliferative agent may comprise etoposide. In yet another aspect, the antiproliferative agent may comprise camptothecin. Furthermore, the antiproliferative agent may comprise cisplatin.

The antiproliferative agent of the invention may comprise an alkylating agent. Alternatively, the antiproliferative agent may be an intercalating agent. In yet another aspect, the antiproliferative agent is a metal coordination complex. The antiproliferative may be a pyrimidine nucleoside. In still another aspect, the antiproliferative agent is a purine nucleoside. In other aspects, the antiproliferative agent is an inhibitor of nucleic acid associated enzymes or an inhibitor of nucleic acid associated proteins.

The antiproliferative agent may be an antimitotic agent. In some aspects, the antiproliferative agent is an antimetabolite. The antiproliferative agent may also be a structural protein agent, an antibiotic, a hormone antagonist or a nucleic acid damaging agent. In still other aspects, the antiproliferative agent is an intercalating agent. The antiproliferative agent may also be a topoisomerase inhibitor, an agent that affects tubulin or a metal coordination complex. In some aspects, the colchicine family member is administered before the administration of said antiproliferative agent. In alternative aspects, the colchicine family member is administered during the administration of said antiproliferative agent. In still other aspects, the colchicine family member is administered after the administration of said antiproliferative agent.

According to some aspects of the invention, the effect on the treated disease with the colchicine family member and antiproliferative composition is greater than that for said antiproliferative agent alone.

The invention also includes a composition comprising a colchicine family member and an antiproliferative agent. The colchicine family member of the composition may comprise colchicine, i.e., (S)-N-(5,6,7,9-tetrahydro-1,2,3,10-tetramethoxy-9-oxobenzo[a]heptalen-7-yl)acetamide, or it may comprise an analog thereof. The invention also includes such compositions wherein the antiproliferative agent comprises etoposide, cisplatin, or camptothecin. Alternatively, the compositions of the invention include an antiproliferative agent such as vinblastine or paclitaxel.

Use of a colchicine family member and an antiproliferative agent in the formulation of a medicament for the treatment of a cellular proliferative disease is also encompassed by the present invention. According to some aspects, the antiproliferative used is vinblastine. According to other aspects, the antiproliferative used is paclitaxel. In still other aspects, the antiproliferative used may be etoposide, camptothecin or cisplatin.

### DETAILED DESCRIPTION OF THE FIGURES

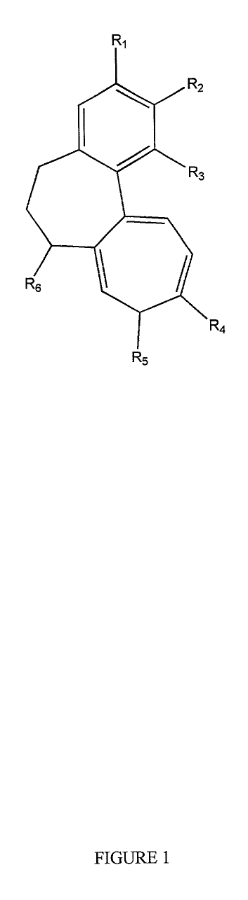

FIG. 1 depicts the general structure of a colchicine family member. R₁ through R₆ represent possible substitution groups.

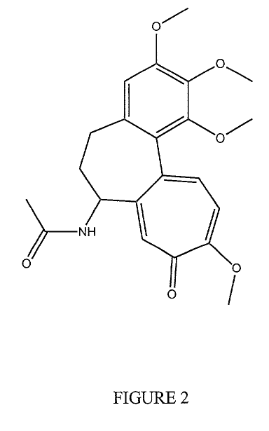

FIG. 2 depicts the chemical structure of colchicine, a colchicine family member described by the chemical name (S)-N-(5,6,7,9-tetrahydro-1,2,3,10-tetramethoxy-9-oxobenzo[a]heptalen-7-yl)acetamide.

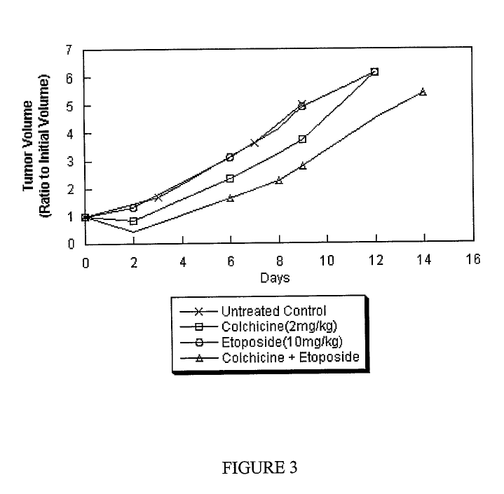

FIG. 3 depicts tumor growth delay, as tumor volume on days after treatment with etoposide, colchicine, or both colchicine and etoposide.

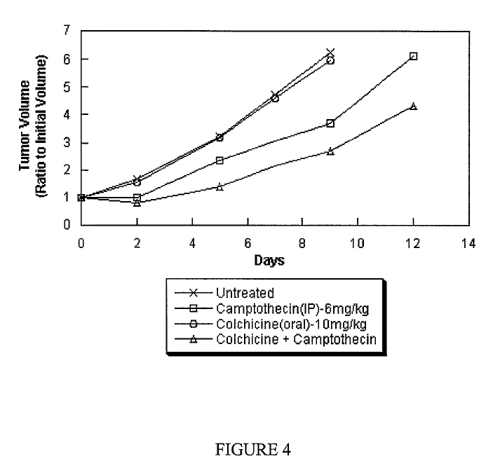

FIG. 4 depicts tumor growth delay, as tumor volume on days after treatment with camptothecin, colchicine, or both colchicine and camptothecin.

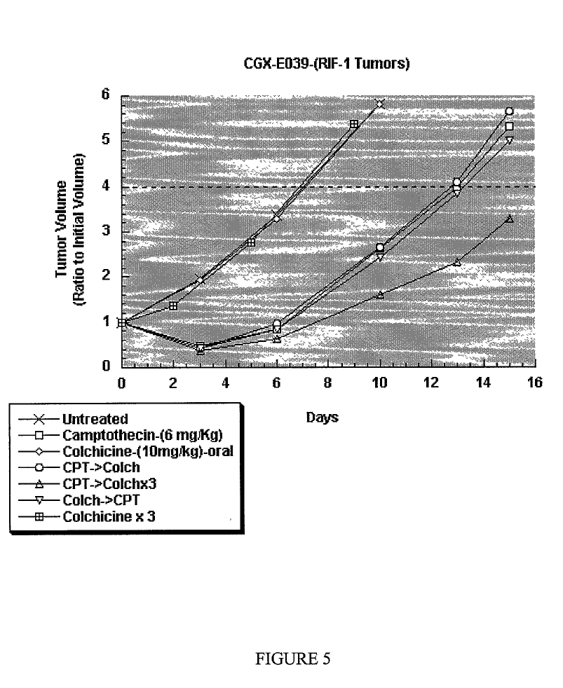

FIG. 5 demonstrates data from an additional experiment with camptothecin. FIG. 5 depicts tumor growth delay, as tumor volume on days after treatment with camptothecin, colchicine, or both colchicine and camptothecin. “Colch×3” or “Colchicine×3” indicates treatment with three doses of colchicine.

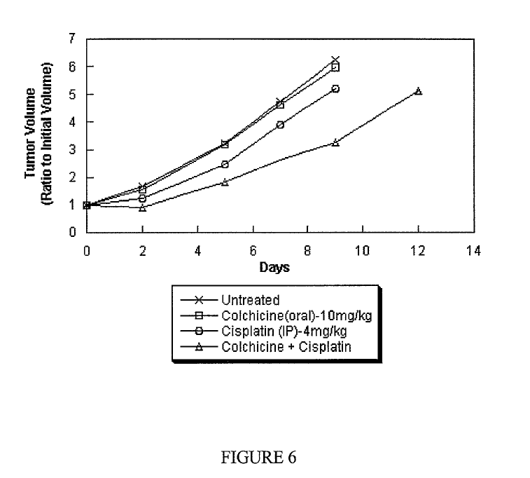

FIG. 6 depicts tumor growth delay, as tumor volume on days after treatment with cisplatin, colchicine, or both colchicine and cisplatin.

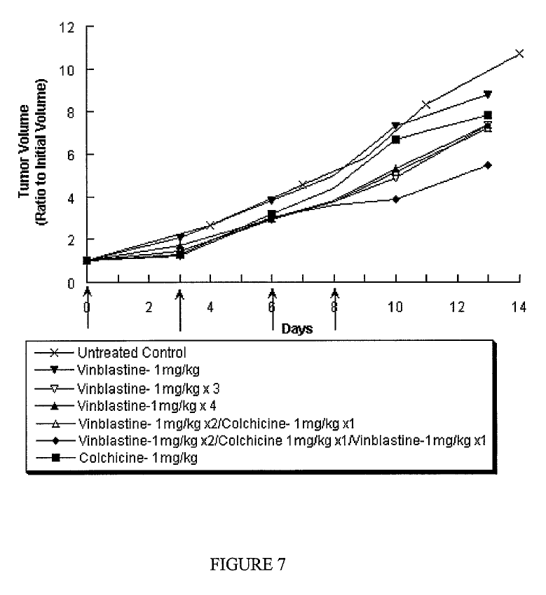

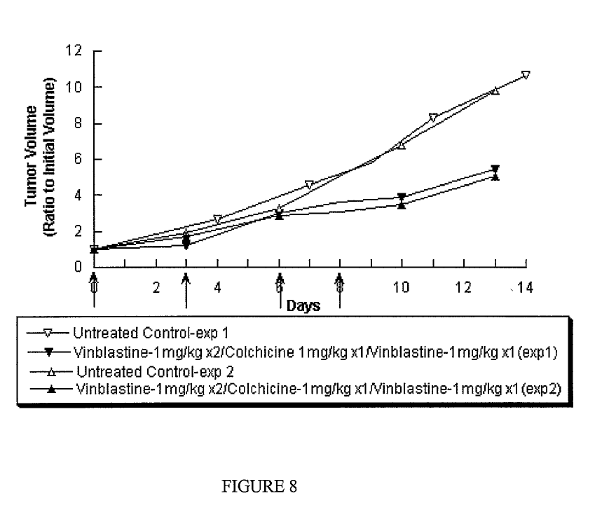

FIGS. 7 and 8 depict tumor growth delay, as tumor volume on days after treatment with vinblastine, colchicine, or both colchicine and vinblastine.

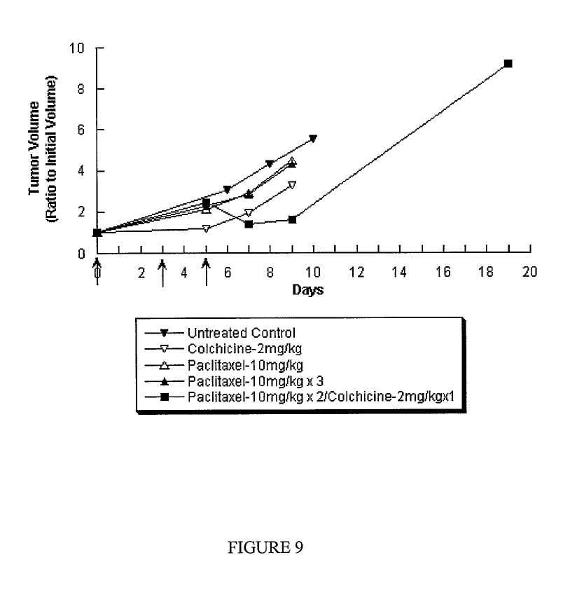

FIG. 9 depicts tumor growth delay, as tumor volume on days after treatment with paclitaxel, colchicine, or both colchicine and paclitaxel.

### DETAILED DESCRIPTION OF THE INVENTION

Methods and compositions are provided for the treatment of a host with a cellular proliferative disease, particularly a neoplasia. In the subject methods, a pharmaceutically acceptable colchicine family member is administered, orally or systemically, in conjunction with an antiproliferative agent to improve the anticancer effects. In a preferred embodiment, the colchicine family member provides a chemopotentiator effect.

The agents are provided in amounts sufficient to modulate a cellular proliferative disease. In one embodiment, modulation of a cellular proliferative disease comprises a reduction in tumor growth. In another embodiment, modulation of a disease comprises inhibition of tumor growth. In another embodiment, modulation of a cellular proliferative disease comprises an increase in tumor volume quadrupling time (described below). In another embodiment, modulation of a cellular proliferative disease comprises a chemopotentiator effect. In another embodiment, modulation of a disease comprises a chemosensitizing effect. In other embodiments, modulation of a disease comprises cytostasis. In still other embodiments, modulation of a disease comprises a cytotoxic effect.

A chemical agent is a “chemopotentiator” when it enhances the effect of a known antiproliferative drug in a more than additive fashion relative to the activity of the chemopotentiator or antiproliferative agent used alone. In some cases, a “chemosensitizing” effect may be observed. This is defined as the effect of use of an agent that if used alone would not demonstrate significant antitumor effects but would improve the antitumor effects of an antiproliferative agent as compared to the antiproliferative agent by itself.

As used herein, “colchicines” or “the colchicine family” includes colchicine and colchicine analogs, generally defined by the chemical structure in FIG. 1.

A preferred colchicine family member is colchicine, (S)-N-(5,6,7,9-tetrahydro-1,2,3,10-tetramethoxy-9-oxobenzo[a]heptalen-7-yl)acetamide, depicted in FIG. 2. Colchicine may also be described by the following chemical and drug names: N-(5,6,7,9-tetrahydro-1,2,3,10-tetramethoxy-9-oxobenzo[a]heptalen-7-yl)-acetamide; N-acetyltrimethylcolchicinic acid methyl ether; 7-acetamido-6,7-dihydro-1,2,3,10-tetramethoxybenzo[a]heptalen-9(5H)-one; 7alphaH-colchicine; colchineos; colchisol; colcin; colsaloid; condylon; colchiceine methyl ether; Colgout; colchicine crystalline.

A colchicine analog is another preferred member of the colchicine family, generally defined by but not limited to the structure depicted in FIG. 1, having substituent changes or substitute groups at one or more of R₁ through R₆. Table 1 lists some possible structures of R₁ through R₆ for colchicine analogs. R group substitutions are typically employed to improve biological activity, enhance pharmaceutical attributes such as bioavailability or stability, or decrease toxicity. In one embodiment, R groups include alkyl substitutions (e.g., methyl, ethyl, propyl etc.). In another embodiment, R groups include an alkoxy (e.g., methoxy, ethoxy, propoxy, butoxy, etc.) substitution. In still other embodiments, R groups include an amino group substitution. In still other embodiments, R groups include a thiol group substitution. Substitutions at R₁ through R₆ are not limited to the above examples, however.

In a preferred embodiment, the substitution or substitutions are of one or more of the substituents corresponding to the R₁ through R₆ positions of colchicine.

TABLE 1

| R group | Substitution | Structure/Length |
|---|---|---|
| R₁₋₃ | Alkyl | −C₁ → C₅ |
| | Alkoxy | −OC₁ → C₅ |
| | Glucoside | −GluO |
| | Hydrogen | −H |
| R₄ | Thiol | −SC₁ → C₅ |
| | Alkyl | −C₁ → C₅ |
| | Alkoxy | −OC₁ → C₅ |
| R₅ | Alkyl | −C₁ → C₅ |
| | Alkoxy | −OC₁ → C₅ |
| | Carbonyl oxygen | =O |
| R₆ | Alkyl | −C₁ → C₅ |
| | Amino | −NH₂ |
| | Nitro | −NO₂ |
| | Cyano | −C≡N |
| | Alkoxy | −OC₁ → C₅ |
| | Thiol | −SH |
| | Acetamide | −NH—CO—CH₃ |

In a preferred embodiment of the invention, the colchicine analog is thiocolchicine (i.e., (S)-N-[5,6,7,9-Tetrahydro-1,2,3-trimethoxy-10-(methylthio)-9-oxobenzo [a]heptalen-7-yl]-acetamide). In another preferred embodiment, the colchicine analog is 3-demethyl thiocolchicine. In still another preferred embodiment, the colchicine analog is thiocolchicoside (i.e., 2-demethoxy-2-glucosidoxythiocolchicine, Colcamyl, Coltramyl, Coltromyl, Coltrax, or Musco-Ril). In another preferred embodiment, the colchicine analog is colchicinamide.

Conventional antiproliferative agents used in the treatment of cancer are broadly grouped as (1) chemical compounds which affect the integrity of nucleic acid polymers by binding, alkylating, inducing strand breaks, intercalating between base pairs or affecting enzymes which maintain the integrity and function of DNA and RNA; (2) chemical agents that bind to proteins to inhibit enzymatic action (e.g., antimetabolites) or the function of structural proteins necessary for cellular integrity (e.g., antitubulin agents). Other chemical compounds that have been identified to be useful in the treatment of some cancers include drugs which block steroid hormone action for the treatment of breast and prostate cancer, photochemically activated agents, radiation sensitizers and protectors.

As used herein, antiproliferative agents are compounds which induce cytostasis or cytotoxicity. “Cytostasis” is the inhibition of cells from growing, while “cytotoxicity” is defined as the killing of cells. Specific examples of antiproliferative agents include: antimetabolites, such as methotrexate, 5-fluorouracil, gemcitabine, cytarabine, pentostatin, 6-mercaptopurine, 6-thioguanine, L-asparaginase, hydroxyurea, N-phosphonoacetyl-L-aspartate (PALA), fludarabine, 2-chlorodeoxyadenosine, and floxuridine; structural protein agents, such as the vinca alkaloids, including vinblastine, vincristine, vindesine, vinorelbine, and paclitaxel; antibiotics, such as dactinomycin, daunorubicin, doxorubicin, idarubicin, bleomycins, plicamycin, and mitomycin; hormone antagonists, such as tamoxifen and luteinizing hormone releasing hormone (LHRH) analogs; nucleic acid damaging agents such as alkylating agents, e.g., mechlorethamine, cyclophosphamide, ifosfamide, chlorambucil, dacarbazine, methylnitrosourea, semustine (methyl-CCNU), chlorozotocin, busulfan, procarbazine, melphalan, carmustine (BCNU), lomustine (CCNU), and thiotepa; fraudulent nucleosides such as purine and pyrimidine analogs; intercalating agents, e.g., doxorubicin, dactinomycin, daurorubicin and mitoxantrone; topoisomerase inhibitors, e.g., etoposide, camptothecin, camptothecin analogs, and teniposide; agents that affect tubulin, e.g., paclitaxel, and metal coordination complexes, e.g., cisplatin and carboplatin.

Of special interest to this invention are compounds that directly affect the integrity of the genetic structure of the cancer cells. Nucleic acid polymers such as DNA and RNA are prime targets for anticancer drugs. Alkylating agents such as nitrogen mustards, nitrosoureas, aziridine containing compounds directly attack DNA. Metal coordination compounds such as cisplatin and carboplatin similarly directly attack the nucleic acid structure resulting in lesions that are difficult for the cells to repair, which in turn, can result in cell death. Other nucleic acid affecting compounds include anthracycline molecules such as doxorubicin, which intercalates between the nucleic acid base pairs of DNA polymers, bleomycin which causes nucleic acid strand breaks, and fraudulent nucleosides such as pyrimidine and purine nucleoside analogs which are inappropriately incorporated into nucleic polymer structures and ultimately cause premature DNA chain termination. Certain enzymes that affect the integrity and functionality of the genome can also be inhibited in cancer cells by specific chemical agents and result in cancer cell death. These include enzymes that affect ribonucleotide reductase (e.g., hydroxyurea, gemcitabine), topoisomerase I (e.g., camptothecin) and topoisomerase II (e.g., etoposide).

The topoisomerase enzymes affect the structure of supercoiled DNA, because most of the functions of DNA require untwisting. Topoisomerase I (top 1) untwists supercoiled DNA, breaking only one of the two strands, whereas topoisomerase II (top2) breaks both.

Topoisomerase I inhibition has become important in cancer chemotherapy through the finding that camptothecin (CPT), an alkaloid of plant origin, is the best known inhibitor of top 1 and is a very potent anticancer agent. CPT is contained in a Chinese tree, Camptotheca acuminata. A number of analogs have become approved for commercial use to treat a number of tumor types. These include CPT-11 (irinotecan) and topotecan.

Topoisomerase II inhibition has also become important in cancer chemotherapy. Chemical families such as the anthracyclines and epipodophyllotoxins play a key role. Drugs from these families (e.g., doxorubicin and etoposide among other chemicals affecting topoisomerase II such as amsacrine, elliptinium, mitoxantrone, azatoxin, genistein, amonafide etc.) form cleavable complexes between the DNA and the topoisomerase II enzyme.

The clinical use of topoisomerase II inhibitors, for example, doxorubicin, amsacrine, etoposide and mitoxantrone, have provided clinical utility to a number of cancers, in particular, solid tumors.

Another agent that targets DNA is cisplatin (cis-diamminedichloroplatinum II), a broadly used anticancer drug. This compound is active against several human cancers including testicular, small-cell lung, bladder, cervical and head and neck cancer.

Also of special interest to this invention are compounds that are known to bind with high affinity to the microtubule protein, tubulin, thereby disrupting microtubule assembly and causing mitotic (cell division) arrest of the proliferating cells. For this reason, “antitubulin agents” are also known as “antimitotic agents,” “microtubule inhibitors” or as “spindle poisons.”

Most of the well characterized antimitotic agents may be arbitrarily divided into three classes: those compounds that competitively inhibit colchicine binding to tubulin and thereby interact with tubulin on the colchicine binding sites (including colchicinoids, podophyllotoxins, steganacins, combretastatins, and amphethinile), those compounds that are believed to share a common binding site on tubulin with the Catharanthus (Vinca) alkaloids (including compounds such as vincristine, vinblastine, maytansinoids, phomopsin A, rhizoxin, the marine antimitotic peptide dolastatin 10) and paclitaxel, a novel taxane diterpenoid isolated from the bark of the Pacific yew which has a very unique antimitotic action. Instead of inhibiting microtubule assembly, paclitaxel and other taxanes promote the formation of stable microtubules that eventually lead to mitotic arrest of proliferating cells.

The following examples are offered by way of illustration and not by way of limitation.

### EXAMPLES

For the examples below, transplantable experimental murine fibrosarcomas (2×10⁵ RIF-1 cells) were grown intradermally in the flanks of 3 month old female C3H mice (Charles River, Holister, Calif.). When the tumors reached a volume of approximately 100 mm³, the mice were randomly assigned to each experimental group (4 mice per group).

Colchicine was obtained from Sigma-Aldrich (St. Louis, Mo.) and was made to the appropriate concentration in water for injection. After the treatments described below, the growth of the tumors was monitored three times per week by caliper measurements of three perpendicular diameters of the tumor. The tumor volume was calculated from the formula:

V=π/6×D₁×D₂×D₃,

where D₁₋₃ are the diameter measurements in mm.

In each Example, the tumors were followed until they reached a size of four times their day zero treatment volume (TVQT), or up to 30 days after treatment, whichever came first. The data is expressed as the “tumor volume quadrupling time” (TVQT) mean and as the “delay.” Mean TVQT is the mean days required for individual tumors to grow to four times the tumor volume at the initial treatment day. The “delay” is the median of days required for a tumor to grow to four times the mean size of the treated group, minus the median of days required to grow to four times the mean size of the control group. The data is also expressed as the ratio of the tumor volume quadrupling time of the treated tumor over the untreated control group (TVQT/CTVQT). Increasing values of this ratio indicate increased antitumor response.

### Example 1

### Chemopotentiation of Etoposide by Colchicine

Etoposide (Sigma-Aldrich, lot. 46H078) was made to the appropriate concentration in DMSO. Etoposide and colchicine were injected systemically (i.e., intraperitoneally, i.p.), in a volume of 100 μl. The experimental compositions were prepared as described in Table 2.

TABLE 2

| Agent | Dose | Solvent | Supplier |
|---|---|---|---|
| Colchicine | 2 mg/kg | Water for Injection | Sigma-Aldrich |
| Etoposide | 10 mg/kg | DMSO | Sigma-Aldrich |

The data is presented in Table 3 below and in FIG. 3.

TABLE 3

| | | | Mean | | | |
|---|---|---|---|---|---|---|
| | | Dose | TVQT ± | TVQT/ | Median | Delay |
| Group | Treatment | (mg/kg) | S.E. | CTVQT | (TVQT) | (Days) |
| 1 | Untreated | — | 7.6 ± 0.3 | — | 7.7 | 0.00 |
| | Control | | | | | |
| 2 | Colchicine | 2 | 9.5 ± 0.2 | 1.2 | 9.3 | 1.65 |
| 3 | Etoposide | 10 | 7.7 ± 0.5 | 1.0 | 7.5 | −0.17 |
| 4 | Colchicine + | 2/10 | 11.3 ± 0.5 | 1.5 | 11.1 | 3.45 |
| | Etoposide | | | | | |

The results of Table 3 indicate that the antiproliferative activity of etoposide is enhanced by the use of colchicine in that a more than additive effect was observed when both compounds were used to treat the tumor bearing mice (group 4) in comparison to the use of etoposide alone (group 3) or colchicine alone (group 2).

### Example 2

### Chemopotentiation of Camptothecin by Colchicine

A. Effect of Single Doses of Colchicine Administered Concurrently with Camptothecin

Camptothecin (Boehringer Ingelheim-Lot 71012) was made to the appropriate concentration in DMSO. Colchicine was given orally in a volume of 100 μl. Camptothecin was injected systemically (i.e., intraperitoneally, i.p.), in a volume of 100 μl. For the treatment of group 3, colchicine was given orally immediately prior to the intraperitoneal injection of camptothecin. The experimental compositions were prepared as described in Table 4.

TABLE 4

| Agent | Dose | Solvent | Supplier |
|---|---|---|---|
| Colchicine | 10 mg/kg | Water for injection | Sigma-Aldrich |
| Camptothecin | 6 mg/kg | DMSO | Boehringer Ingelheim |

The data is presented in Table 5 below and in FIG. 4.

TABLE 5

| | | Dose | Mean | TVQT/ | Median | |
|---|---|---|---|---|---|---|
| Group | Treatment | (mg/kg) | TVQT ± S.E. | CTVQT | (TVQT) | Delay (Days) |
| 1 | Untreated Control | — | 6.3 ± 0.3 | — | 6.3 | 0.00 |
| 2 | Colchicine | 10 | 6.4 ± 0.3 | 1.0 | 6.3 | 0.02 |
| 3 | Camptothecin | 6 | 9.4 ± 0.5 | 1.5 | 9.9 | 3.60 |
| 4 | Colchicine + | 10/6 | 10.9 ± 0.2 | 1.7 | 10.9 | 4.60 |
| | Camptothecin | | | | | |

The results of Table 5 indicate that the antiproliferative activity of camptothecin is enhanced by the use of colchicine in that a more than additive effect was observed when both compounds were used to treat the tumor bearing mice (group 4) in comparison to the use of camptothecin alone (group 3) or colchicine alone (group 2).

B. Effect of Single or Multiple Doses of Colchicine Administered in Mixed Sequence

FIG. 5 demonstrates the results of an additional experiment using camptothecin and colchicine. In FIG. 5, the arrow indicates a one hour interval separating treatment with each agent. The experimental compositions were prepared as described above. In two treatment groups, colchicine was administered orally in three doses (“colch×3” or “colchicine×3”). When three dosages of colchicine were administered, the first was given one hour after the administration of camptothecin (day 0), the second on day 1 and the third on day 2.

The data presented in Table 6 and FIG. 5 demonstrate that administering three doses of colchicine to mice did not delay tumor growth significantly as compared to administering a single dose. However, administering camptothecin followed by three doses of colchicine delayed tumor growth more than did camptothecin alone, colchicine alone, or a one time dose of camptothecin followed by colchicine.

TABLE 6

| | # of | | Dose | Days to 4× | | | |
|---|---|---|---|---|---|---|---|
| Treatment | Tumors | Route | (mg/kg) | (Ave ± SE) | T/C | Median | Delay |
| Untreated | 8 | — | — | 7.5 ± 0.6 | — | 7.3 | — |
| CPT | 8 | IP | 6 | 12.9 ± 0.5 | 1.7 | 12.8 | 5.45 |
| Colchicine | 8 | oral | 10 | 7.7 ± 0.4 | 1.0 | 7.6 | 0.32 |
| CPT→colch | 8 | IP/oral | 6→10 | 13.0 ± 0.7 | 1.7 | 12.5 | 5.18 |
| CPT→colch × 3 | 6/8 | IP/oral | 6→10 × 3 | 16.5 ± 1.1 | 2.2 | 16.5 | 9.15 |
| (D-0,1,2) | | | | | | | |
| colch→CPT | 8 | oral/IP | 10→6 | 13.5 ± 0.8 | 1.8 | 13.4 | 6.08 |
| colchicine × 3 | 8 | oral | 10 × 3 | 7.2 ± 0.3 | 1.0 | 7.3 | −0.04 |
| (D-0,1,2) | | | | | | | |

The arrow → represents a 1 hour interval
D represents day of treatment.

### Example 3

### Chemopotentiation of Cisplatin by Colchicine

Cisplatin (David Bull Laboratories-Mulgrave, Australia, lot. 5201844x) was made to the appropriate concentration in water for injection. Colchicine was given orally in a volume of 100 μl. Cisplatin was injected systemically (i.e., intraperitoneally, i.p.), in a volume of 100 μl. For the treatment of group 3, colchicine was given orally immediately prior to the intraperitoneal injection of cisplatin. The experimental compositions were prepared as described in Table 7.

TABLE 7

| Agent | Dose | Solvent | Supplier |
|---|---|---|---|
| Colchicine | 2 mg/kg | Water for Injection | Sigma-Aldrich |
| Cisplatin | 10 mg/kg | Water for injection | David Bull Labs |

The data is presented in Table 8 below and in FIG. 6.

TABLE 8

| | | Dose | TGD ± | TGD/ | Median | Delay |
|---|---|---|---|---|---|---|
| Group | Treatment | (mg/kg) | S.E. | CTGD | (TGD) | (Days) |
| 1 | Untreated | — | 6.3 ± 0.3 | — | 6.3 | 0.00 |
| | Control | | | | | |
| 2 | Colchicine | 10 | 6.4 ± 0.3 | 1.0 | 6.3 | 0.02 |
| 3 | Cisplatin | 4 | 7.4 ± 0.3 | 1.2 | 7.7 | 1.45 |
| 4 | Colchicine + | 10/4 | 12.2 ± 1.7 | 1.9 | 9.9 | 3.59 |
| | Cisplatin | | | | | |

The results of Table 8 indicate that the antiproliferative activity of cisplatin is enhanced by the use of colchicine in that a more than additive effect was observed when both compounds were used to treat the tumor bearing mice (group 4) in comparison to the use of cisplatin alone (group 3) or colchicine alone (group 2).

### Example 4

### Enhancement of Tubulin-Targeted Cell Killing by Colchicine After and During Treatment with Vinblastine

The experimental compositions were prepared as described in Table 9.

TABLE 9

| Agent | Dose | Solvent | Supplier |
|---|---|---|---|
| Colchicine | 1 mg/kg | Water for injection | Sigma |
| Vinblastine | 1 mg/kg | Water for injection | Faulding (Elizabeth, NJ) |

Vinblastine was obtained from Faulding (Elizabeth, NJ) and was made to the appropriate concentration in water for injection. The compositions (1 mg/kg of either colchicine or vinblastine) were injected systemically (i.e., intraperitoneally, i.p.), in a volume of 100 μl. For group 4, vinblastine treatments were given three times, on days 0, 3, and 6 (Day 0 is the first day of treatment). For group 6, vinblastine treatments were given twice, on days 0 and 3, and colchine was given on day 6. For group 5, four vinblastine treatments were given on days 0, 3, 6 and 8. For group 7, vinblastine treatments were given on days 0 and 3, a colchicine treatment was given on day 6, and a third vinblastine treatment was given on day 8.

The data are presented in Table 10 below and in FIG. 7.

TABLE 10

| | | Mean TVQT ± | TVQT/ | Median | Delay |
|---|---|---|---|---|---|
| Group | Treatment | S.E. | CTVQT | (TVQT) | (Days) |
| 1 | Untreated Control | 6.3 ± 0.3 | — | 6 | 0.00 |
| 2 | Colchicine | 7.2 ± 0.5 | 1.1 | 7.2 | 1.15 |
| 3 | Vinblastine-1× | 6.6 ± 0.4 | 1.0 | 6.2 | 0.11 |
| 4 | Vinblastine-3× | 8.9 ± 0.9 | 1.4 | 7.8 | 1.78 |
| 5 | Vinblastine-4× | 8.4 ± .06 | 1.2 | 8.6 | 1.67 |
| 6 | Vinblastine-2×/ | 9.1 ± 1.0 | 1.4 | 8.2 | 2.15 |
| | Colchicine-1× | | | | |
| 7 | Vinblastine-2×/ | 10.1 ± 0.9 | 1.6 | 10.1 | 4.01 |
| | Colchicine-1×/ | | | | |
| | Vinblastine-1× | | | | |

The results of Table 3 indicate that the antiproliferative activity of vinblastine can be restored by the use of colchicine after vinblastine resistance has been achieved. As demonstrated in the graph in FIG. 7, vinblastine given 3 or 4 times, and vinblastine given twice followed by one treatment of colchicine gave the same tumor growth delay curves. However, when vinblastine was given after colchicine, as in group 7, the tumor growth delay curve is deflected to indicate renewed sensitivity to vinblastine. This result was repeated as demonstrated in FIG. 8.

### Example 5

### Enhancement of Tubulin-Targeted Cell Killing by Colchicine After and During Treatment with Paclitaxel

The experimental compositions were prepared as described in Table 11.

TABLE 11

| Agent | Dose | Solvent | Supplier |
|---|---|---|---|
| Colchicine | 1 mg/kg | Water for injection | Sigma |
| Paclitaxel | 10 mg/kg | saline | Mead-Johnson |

Paclitaxel was obtained prediluted at 1 mg/ml in a cremaphor/ethanol solution. Colchicine was given at a dose of 2 mg/kg and paclitaxel was given, for each injection, at a dose of 10 mg/kg. For group 4, a total of three paclitaxel injections were given, one each on days 0, 3 and 5. The compositions were injected systemically (i.e., intraperitoneally, i.p.), in a volume of 100 μl.

The data are presented in Table 12 below and in FIG. 9.

TABLE 12

| | | Mean TVQT ± | TVQT/ | Median | Delay |
|---|---|---|---|---|---|
| Group | Treatment | S.E. | CTVQT | (TVQT) | (Days) |
| 1 | Untreated Control | 7.5 ± 0.3 | — | 7.4 | 0.00 |
| 2 | Colchicine | 11.3 ± 0.6 | 1.5 | 10.8 | 3.34 |
| 3 | Paclitaxel | 9.1 ± 0.7 | 1.2 | 8.1 | 0.67 |
| 4 | Paclitaxel-3× | 10.8 ± 1.9 | 1.4 | 9.0 | 1.59 |
| 5 | Paclitaxel-3×/ | 13.8 ± 0.5 | 1.8 | 13.8 | 6.33 |
| | Colchicine-1× | | | | |

The results of Table 12 and FIG. 9 indicate that paclitaxel treatment is slightly more effective in the RIF-1 model when given 3 times than when given once. However, tumors treated with paclitaxel are sensitive to colchicine treatment such that an improvement in anti-tubulin targeted therapy can be achieved.

We claim:

1. A method of treatment of a host with a cellular proliferative disease, comprising contacting said host with a colchicine family member and an antiproliferative agent, each in an amount sufficient to modulate said cellular proliferative disease.

2. The method according to claim 1, wherein said colchicine family member comprises colchicine, (S)-N-(5,6,7,9-tetrahydro-1,2,3,10-tetramethoxy-9-oxobenzo[a]heptalen-7-yl)acetamide.

3. The method according to claim 1, wherein said colchicine family member comprises a colchicine analog.

4. The method according to claim 3 wherein said colchicine analog is thiocolchicoside, 2-demethoxy-2-glucosidoxythiocolchicine.

5. The method according to claim 1 wherein said antiproliferative agent comprises an agent that interacts with nucleic acids.

6. The method according to claim 1 wherein said antiproliferative agent comprises an agent that interacts with tubulin targets.

7. The method according to claim 6 wherein said antiproliferative agent comprises taxanes, vinca alkaloids or a tubulin targeted agent.

8. The method according to claim 1 wherein said antiproliferative agent comprises paclitaxel.

9. The method according to claim 1 wherein said antiproliferative agent comprises vinblastine.

10. The method of claim 1 wherein said antiproliferative agent comprises alkylating agents, intercalating agents, metal coordination complexes, pyrimidine nucleosides, purine nucleosides, inhibitors of nucleic acid associated enzymes, or inhibitors of nucleic acid associated proteins.

11. The method of claim 1 wherein said antiproliferative agent comprises etoposide.

12. The method of claim 1 wherein said antiproliferative agent comprises camptothecin.

13. The method according to claim 1 wherein said antiproliferative agent comprises cisplatin.

14. A method according to claim 1 when said colchicine family member is administered before the administration of said antiproliferative agent.

15. A method according to claim 1 when said colchicine family member is administered during the administration of said antiproliferative agent.

16. A method according to claim 1 when said colchicine family member is administered after the administration of said antiproliferative agent.

17. The method of claim 1 wherein the effect on said disease with said composition is greater than that for said antiproliferative agent alone.

18. A composition comprising a colchicine family member and an antiproliferative agent.

19. The composition of claim 18 wherein said colchicine family member comprises: colchicine, (S)-N-(5,6,7,9-tetrahydro-1,2,3,10-tetramethoxy-9-oxobenzo[a]heptalen-7-yl)acetamide.

20. The composition of claim 18 wherein said colchicine family member comprises a colchicine analog.

21. The composition of claim 18 wherein said colchicine analog is thiocolchicoside, 2-demethoxy-2-glucosidoxythiocolchicine.

22. The composition of claim 18, wherein said antiproliferative agent comprises etoposide, cisplatin, or camptothecin.

23. The composition of claim 18, wherein said antiproliferative agent comprises vinblastine or paclitaxel.

24. Use of a colchicine family member and an antiproliferative agent in the formulation of a medicament for the treatment of a cellular proliferative disease.
